(19) United States
(12) Patent Application Publication
(10) Pub. No.: US 2013/0261425 A1
(43) Pub. Date: Oct. 3, 2013

(54) PROBE AND OBJECT INFORMATION ACQUISITION APPARATUS USING THE SAME

(71) Applicant: CANON KABUSHIKI KAISHA, Tokyo (JP)

(72) Inventors: Kazunari Kawabata, Kawasaki-shi (JP); Koichiro Nakanishi, Tokyo (JP)

(73) Assignee: CANON KABUSHIKI KAISHA, Tokyo (JP)

(21) Appl. No.: 13/795,837

(22) Filed: Mar. 12, 2013

(30) Foreign Application Priority Data
Mar. 31, 2012 (JP) 2012-083417
Feb. 28, 2013 (JP) 2013-040132

(51) Int. Cl. A61B 5/00

(52) U.S. Cl. CPC A61B 5/0095
USPC 600/407

(57) ABSTRACT

An optical reflection member in a probe has a gas barrier layer for compensating for a high gas transparency of a support layer, thereby avoiding effects of deterioration of adhesion between an optical reflection layer and a support layer due to gas inflow, and reducing exfoliation. The probe includes an element having at least one cell in which is supported a vibration film containing one out two electrodes that define a space therebetween, in a manner allowing the acoustic wave to vibrate the film. An optical reflection layer is provided closer to the object than the element is, a support layer 104 supports the optical reflection layer, and a gas barrier layer that has a higher gas barrier property than the support layer is provided on at least one of the surfaces of the support layer.

### BACKGROUND OF THE INVENTION

1. Field of the Invention

The present invention relates to a probe that is used as a photoacoustic probe and includes a capacitive electromechanical transducer having an optical reflection member, and an object information acquisition apparatus using the same.

2. Description of the Related Art

In recent years, ultrasonic diagnosis, particularly, photoacoustic tomography (PAT) using photoacoustics, has attracted attention as a technique of detecting diseases at an early stage. The technique is noninvasive to a living body and visualizes information in the living body using near-infrared light with a high transmittance. When a living body is irradiated with near-infrared light, the living body absorbs light energy and instantaneously thermally expands to thereby emit acoustic waves. The technique detects the acoustic waves and images the inside of the living body. In this specification, the acoustic waves are any of sound waves, ultrasonic waves, and photoacoustic waves. For instance, the photoacoustic waves are caused by irradiating the inside of an object with light (electromagnetic waves), such as visible or infrared light. Hereinafter, a term of “ultrasound” may be used as typical acoustic waves.

FIG. 7 is a schematic diagram of photoacoustic tomography. A diagnostic target (object) 504, such as a living body, is irradiated with a laser beam 502. Ultrasound 506 caused in the object 504 is detected by a probe 508. Ultrasound significantly attenuates in air, and is strongly reflected by an interface of substances having different acoustic impedances. Thus, an acoustic medium 500 having an acoustic impedance equivalent to that of the object and the probe is typically filled between the object 504 and the probe 508. Diagnosis is performed on a desired region in the object 504 by scanning the laser beam 502 and the probe 508 in synchronous scan with each other. When the laser beam 502 comes off the object 504, the probe 508 is directly irradiated with the beam to cause large acoustic noise. The noise may affect diagnosis. In a certain configuration, the laser beam 502 is incident on the same side as that of the probe 508. Even in this case, when scattered light enters the probe 508 and is absorbed, noise may occur. Thus, an optical reflection member reflecting the laser beam 502 can be provided on the surface of the probe 508.

Required characteristics of the optical reflection member includes: 1) a high reflectance in a wavelength region of light to be used; 2) a high transparency to a signal (ultrasound) caused from an object; and 3) acoustic impedance consistency with an ambient acoustic medium. A metal film has a high reflectance to light but has a high acoustic impedance. In consideration of acoustic impedance consistency, the metal film is required to have a thickness of about 1/30 or less of the wavelength of sound in the metal. IEEE Transactions on Medical Imaging, Vol. 24, NO. 4, 2005 pp. 436-440 describes an optical reflection member in which a resin foil is coated with aluminum having a thickness of 8 μm. This thickness is about 1/100 of the wavelength of 10 MHz ultrasound in aluminum (642 μm), and thus sufficiently thin. Accordingly, the thickness does not seem to cause a problem in terms of acoustic impedance. However, the document does not include detailed description on the resin part, which is a support layer for the aluminum. The resin part requires the characteristics 2) and 3). In IEEE Transactions on Medical Imaging, Vol. 24, NO. 4, 2005 pp. 436-440, a sensor including a piezoelectric element, such as PZT, is used as an ultrasound sensor. In recent years, a capacitive micromachined ultrasonic transducer (CMUT), which is a capacitive electromechanical transducer, has also been widely used.

The CMUT has an acoustic impedance close to that of a living body. This impedance basically negates the need of an impedance consistency layer, and the CMUT has a wide band. Accordingly, the CMUT is particularly suitable for an ultrasound sensor for diagnosing a living body. Ultrasound significantly attenuates in air, which has extremely small acoustic impedance; ultrasound is substantially 100% reflected by the interface between air and another substance. Thus, an medium (acoustic medium) that is typically safe for a human body and has an acoustic impedance close to that of a living body (an acoustic impedance of about 1.5×10⁶ [kg·m⁻²·s⁻¹]) is inserted between the living body and the ultrasound sensor. Water (with an acoustic impedance of about 1.5×10⁶ [kg·m⁻²·s⁻¹]) and polyethylene glycol (with an acoustic impedance about 1.8×10⁶ [kg·m⁻²·s⁻¹]) can be used. A material having a low acoustic impedance (e.g., about 2×10⁶ [kg·m⁻²·s⁻¹] or less) close to that of the acoustic medium of a support layer (the resin foil in IEEE Transactions on Medical Imaging, Vol. 24, NO. 4, 2005 pp. 436-440) of an optical reflection member can be used. For instance, polycarbonate resin has an acoustic impedance of about 2.6×10⁶ [kg·m⁻²·s⁻¹], and sometimes causes reflection of ultrasound due to acoustic impedance inconformity, and reduction in sensitivity and a band degradation. Accordingly, polycarbonate resin is not suitable. Japanese Patent Application Laid-Open No. 2010-75681 discloses the optical reflection member in which polymethylpentene resin (acoustic impedance of about 1.8×10⁶ [kg·m⁻²·s⁻¹]) is coated with the metal film, as an optical reflection member satisfying suitable conditions.

### SUMMARY OF THE INVENTION

According to the above reasons, the optical reflection member in the photoacoustic probe can be a combination of a support layer made of a low acoustic impedance (2×10⁶ [kg·m⁻²·s⁻¹] or less) and an optical reflection layer, such as a metal thin film. However, in the case of actually using this combination as the optical reflection member, the inventors of the present invention have found the following points. That is, resin of olefin series used as a support layer has a high gas transparency due to the low density, and allows gas to flow into the adhesive interface, thereby degrading the adhesive property. Even if a metal thin film to be an optical reflection layer is formed on the surface of resin, there is a possibility that gas flowing from the side of resin changes the state of the surface to degrade the adhesive property of the optical reflection layer, thereby exfoliating the layer.

To solve the problem, the present invention has an object to provide a probe that includes an optical reflection member having a sufficient adhesive property between an optical reflection layer and a support layer.

A probe of the present invention is a probe receiving an acoustic wave from an object, including: an element having at least one cell in which a vibration film containing one electrode out of two electrodes that are provided so as to interpose a space therebetween is supported in a manner allowed to vibrate owing to the acoustic wave; an optical reflection layer that is provided closer to the object than the element is; a support layer that is provided closer to the element than the optical reflection layer is, and supports the optical reflection layer; and a gas barrier layer that is provided on at least one of a surface of the support layer closer to the optical reflection layer and a surface of the support layer closer to the element and has a higher gas barrier property than the support layer.

Further features of the present invention will become apparent from the following description of exemplary embodiments with reference to the attached drawings.

### BRIEF DESCRIPTION OF THE DRAWINGS

FIG. 1 is a sectional view of an example of a photoacoustic probe of the present invention.

FIGS. 2A, 2B and 2C are sectional views illustrating an example of a flow of steps of manufacturing a photoacoustic probe of the present invention.

FIGS. 3A, 3B and 3C are sectional views illustrating a flow of steps of manufacturing a photoacoustic probe of the present invention having a different layer configuration.

FIGS. 4A, 4B, 4C and 4D are sectional views illustrating a flow of steps of manufacturing a photoacoustic probe of the present invention having a different layer configuration.

FIG. 5A is a top plan view of the probe. FIG. 5B is a sectional view of a probe using a capacitive electromechanical transducer (sacrifical layer type) taken along line 5B-5B.

FIG. 6 is a sectional view of a probe using a capacitive electromechanical transducer (bonding type).

FIG. 7 is a diagram schematically illustrating photoacoustic tomography.

FIG. 8 is a diagram schematically illustrating the photoacoustic probe of the present invention.

FIG. 9 is a diagram illustrating an object information acquisition apparatus using the probe of the present invention.

### DESCRIPTION OF THE EMBODIMENTS

Preferred embodiments of the present invention will now be described in detail in accordance with the accompanying drawings.

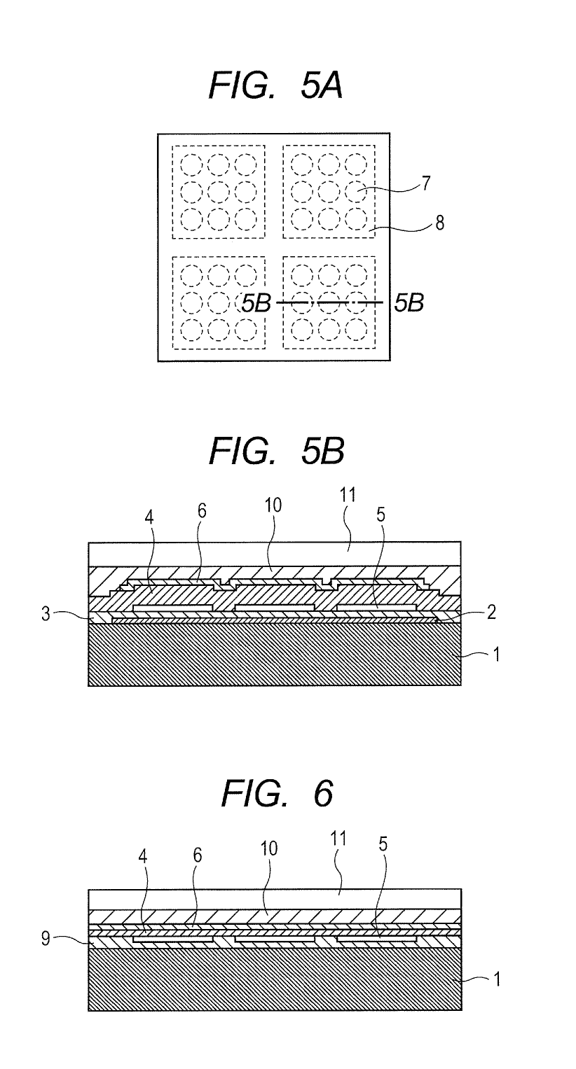

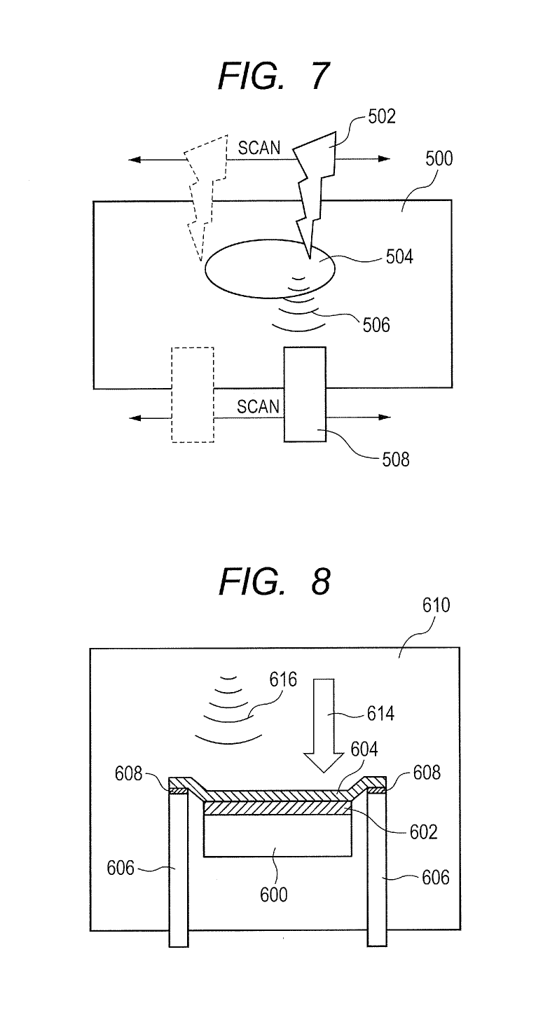

A probe of this embodiment includes a capacitive electromechanical transducer as a detection unit of receiving acoustic waves from an object. An optical reflection member provided on a vibration film (i.e., on a side that is closer to an object than the element is) includes a support layer, an optical reflection layer and a gas barrier layer having a higher gas barrier property than the support layer. The gas barrier layer is provided on at least one of a side of the support layer facing the optical reflection layer and the other side of the support layer facing the element. In such a configuration, the gas barrier property is a transparency of gas (typically, oxygen). A low transmittance represents a high gas barrier property. More specifically, in a state where the gas barrier layer is formed on the support layer, the oxygen transmittance may suitably be 1×10⁻¹⁵ cm³·cm/(cm²·s·Pa) or lower. The acoustic wave transparency is a transmittance of acoustic waves. A high acoustic wave transparency represents a property allowing, for instance, at least 90% of acoustic waves to transmit. The optical reflectivity is a reflectance of light. A high optical reflectivity represents, for instance, a property that reflects at least 80%, more suitably 90%, of light used in a wavelength band of the light. For instance, a cell of the electromechanical transducer includes: a second electrode formed, via a space, on a first electrode formed in contact with a substrate; a vibration film on which the second electrode is provided; and a vibration film supporter that supports the vibration film such that a space is formed between the first electrode and the vibration film. The cell can be fabricated according to a method of manufacturing any of types called a sacrificial layer type and a bonding type. An example of FIGS. 5A and 5B, which will be described later, includes a structure that can be fabricated according to the method of manufacturing a sacrificial layer type. An example of FIG. 6, which will be described later, includes a structure that can be fabricated according to the method of manufacturing the bonding type. The probe of the present invention, a light source and a data processing device can configure an object information acquisition apparatus. Here, the probe receives acoustic waves caused by irradiation of an object with light emitted from the light source, converts the waves into an electric signal. The data processing device acquires information on the object using the electric signal.

Next, an example of a photoacoustic probe according to the present invention will be described. FIG. 8 is a schematic diagram of the photoacoustic probe. The probe includes: a device substrate 600 including a CMUT (i.e., the element illustrated in FIGS. 5A and 5B) as an ultrasound sensor; an acoustic impedance matching layer 602 having functions of protecting the CMUT and transmitting ultrasound 616; and an optical reflection member 604 for reflecting a laser beam 614 at a high reflectance. These components are accommodated in a case 606. The case 606 and the optical reflection member 604 are sealed to each other with adhesive 608, which prevents an acoustic medium 610 from entering the case 606.

The capacitive electromechanical transducer included in the probe of the embodiment of the present invention will be described. FIGS. 5A and 5B illustrate an example of the probe using a CMUT including an element having a plurality of cells. FIG. 5A is a top plan view. FIG. 5B is a sectional view of FIG. 5A taken along line 5B-5B. The probe includes a plurality of elements 8 including cells 7. In FIGS. 5A and 5B, each of four elements 8 includes nine cells 7. However, only if at least one cell is included in each element 8, the number of cells is arbitrary.

As illustrated in FIG. 5B, a cell 7 in this embodiment includes a substrate 1, a first electrode 2, an insulation film 3 on the first electrode 2, a vibration film 4 provided on the insulation film 3 via a space 5 (cavity), and a second electrode 6 on the vibration film 4. In the cell 7, a vibration film including one of the two electrodes interposing the space is supported in a manner allowing the vibration film to vibrate. The substrate 1 is made of Si. Instead, this substrate may be an insulating substrate made of glass. The first electrode 2 is a metal thin film made of any of titanium and aluminum. In the case where the substrate 1 is made of silicon with a low resistance, the substrate itself can serve as the first electrode 2. The insulation film 3 can be formed by stacking a thin film made of silicon oxide. A vibration film supporter 9 supporting the vibration film 4 in a manner allowing this film to vibrate is formed by stacking a thin film made of silicon nitride. The second electrode can be formed of a metal thin film made of any of titanium and aluminum. In this specification, the vibration film at a membrane part made of one of a silicon nitride film and a single crystal silicon film, and the second electrode may be collectively called the vibration film.

The probe of this embodiment can be formed using the method of manufacturing a bonding type. A cell 7 having the bonding type configuration illustrated in FIG. 6 includes a vibration film 4 provided on a silicon substrate 1 via a space 5, a vibration film supporter 9 supporting the vibration film 4 in a manner allowing this film to vibrate, and a second electrode 6. Here, the silicon substrate 1 having a low resistance also serves as the first electrode. Instead, the substrate may be an insulation glass substrate. In this case, a metal thin film (one of titanium and aluminum) to serve as the first electrode 2 is formed on the substrate 1. The vibration film 4 is formed of a junction silicon substrate. Here, the vibration film supporter 9 is made of silicon oxide. Instead, this supporter may be formed by stacking a thin film made of silicon nitride. The second electrode 6 is formed of a metal thin film made of aluminum. FIGS. 5 and 6 illustrate an acoustic impedance matching layer 10, and optical reflection member 11 including a gas barrier layer.

A principle of driving the probe of this embodiment will be described. The cell is formed of the first electrode 2 and the vibration film that interpose the space 5. Accordingly, to receive acoustic waves, a direct current voltage is applied to one of the first electrode 2 and the second electrode 6. When the acoustic waves are received, the acoustic waves vibrate the vibration film to change the distance (height) of the space. Accordingly, the capacitance between the electrodes is changed. The change in capacitance is detected from one of the first electrode 2 and the second electrode 6, thereby allowing the acoustic waves to be detected. The element can also transmit acoustic waves by applying an alternating voltage to one of the first electrode 2 and the second electrode 6 to vibrate the vibration film.

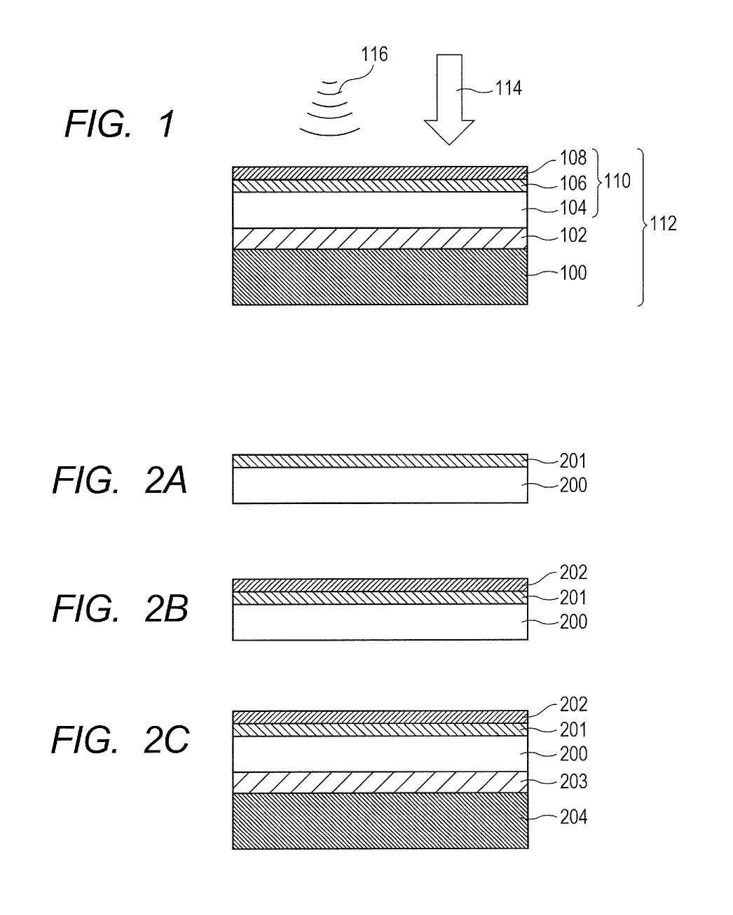

Referring to FIG. 1, the layer configuration on the electromechanical transducer, which characterizes the present invention, will be further described in detail. FIG. 1 is a sectional view illustrating the probe. FIG. 1 illustrates a substrate (CMUT substrate) 100 including a CMUT element, an acoustic impedance matching layer 102 formed between the CMUT substrate 100 and a support layer 104, a gas barrier layer 106, an optical reflection layer 108, and an optical reflection member 110 including the support layer 104, the gas barrier layer 106 and the optical reflection layer 108. The CMUT substrate 100, the acoustic impedance matching layer 102 and the optical reflection member 110 configure a photoacoustic probe 112. The photoacoustic probe 112 is typically used in an acoustic medium having an acoustic impedance close to that of a living body. The acoustic medium is, for instance, one of water and polyethylene glycol.

The CMUT substrate 100 typically has a configuration in which capacitance type sensors are two dimensionally arranged. The sensor includes a membrane made of one of Si and SiN on a cavity formed on the Si substrate and is called a cell. The arrangement configuration is appropriately selected according to the usage thereof. The acoustic impedance matching layer 102 has a function of protecting the membrane on the CMUT substrate 100 and a function of efficiently transmitting ultrasound 116 from an optical reflection member 110 to the CMUT substrate 100. That is, the acoustic impedance matching layer 102 is formed on the vibration film, and can suitably be made of what has a low Young's modulus that does not largely change mechanical characteristics, such as the spring constant of the membrane. More specifically, a suitable Young's modulus is 50 MPa or less. The Young's modulus of 50 MPa or less alleviates adverse effects on the vibration film due to the stress of optical reflection layer 108. Since the stiffness (Young's modulus) is sufficiently low, the substantial mechanical property of the vibration film 7 is not changed. Furthermore, the acoustic impedance matching layer 102 is suitably made of material having an acoustic impedance equivalent to that of the membrane. More specifically, the suitable acoustic impedance ranges from 1 MRayls to 2 MRayls, inclusive (1 MRayls=1×10⁶ kg·m⁻²·s⁻¹). The material suitably employed for the acoustic impedance matching layer 102 is material having a small adverse effect on the mechanical property of the membrane of the CMUT. For instance, silicone rubber of bridged polydimethylsiloxane (PDMS) is suitable. There are various PDMSs, which include fluorosilicone series in which a part thereof is replaced with fluorine, and into which an additive, such as a filler, is mixed. A PDMS is appropriately selected in consideration of consistency of acoustic property with that of the acoustic medium and the optical reflection member.

The optical reflection layer 108 is for reflecting a laser beam 114, and provided closer to an object than the element 8 is. More specifically, this layer reflects light emitted on the object and the scattered light. In the case of diagnosing a living body, a near-infrared region of wavelengths from about 700 to 1000 nm is often used as the laser beam 114. The optical reflection layer 108 is suitably a metal film having a high reflectance (suitably, 80%, and more suitably 90%) in the wavelength region (e.g., 700 to 1000 nm) to be used. More specifically, a film of one of Au, Ag and an alloy thereof can suitably be used. The thickness of the optical reflection layer 108 can be 10 μm or less in consideration of the acoustic impedance. For instance, in the case of Au, the acoustic impedance is about 63×10⁶ [kg·m⁻²·s⁻¹], which is high. Accordingly, the thickness of the layer is required to be sufficiently reduced to prevent reflection of ultrasound due to inconformity of the acoustic impedance. The thickness can thus be 1/30 or less of the wavelength of ultrasound in the material. In the case of Au, the thickness of the layer is preferably 10 μm or less, and more preferably 0.1 μm to 1 μm inclusive in consideration of reduction in material cost. Moreover, a dielectric multilayer film is formed on the metal film made of Au can be formed to configure a layered structure, thereby allowing the reflectance to be further improved. The optical reflection layer may be a dielectric multilayer film.

The support layer 104 is a layer for supporting such an optical reflection layer, and provided closer to the element 8 than the optical reflection layer 108 is. The support layer 104 is suitably made of material having an acoustic impedance equivalent to that of an ultrasound transmitting medium and favorable ultrasound transparency. The optical reflection layer 108 can be formed directly on the acoustic impedance matching layer 102. However, this reflection layer can suitably be formed on the support layer 104. The acoustic impedance matching layer 102 is made of material having a low Young's modulus. Accordingly, in the case of forming the optical reflection layer 108 directly on the acoustic impedance matching layer, there is a possibility that the stress from the optical reflection layer deforms the acoustic impedance matching layer. The acoustic impedance matching layer 102 is made of material having a low Young's modulus. It is therefore difficult to reduce the surface roughness. Furthermore, it is difficult to increase the reflectance of the optical reflection layer on the acoustic impedance matching layer. Thus, the optical reflection layer 108 can be suitably formed on the support layer 104 having a higher stiffness than the acoustic impedance matching layer 102. More specifically, the acoustic impedance of the support layer 104 can be about between 1 and 5 MRayls, inclusive. The Young's modulus of the support layer 104 is larger (higher) than that of the acoustic impedance matching layer 102, and more specifically, between 100 MPa and 20 GPa, inclusive. The acoustic impedance of the support layer 104 is configured close to the value of the acoustic impedance of the acoustic impedance matching layer 102, thereby allowing the amount of reflection of acoustic waves to be reduced at the interface between the support layer 104 and the acoustic impedance matching layer 102. Resin of olefin series is suitable for material having an acoustic property close to a living body. For instance, polymethylpentene resin and polyethylene can suitably be used. In consideration of forming the optical reflection layer 108 and adhesion to the CMUT substrate 100, appropriate flexibility is suitable. What have a thickness of about 10 to 150 μm can suitably be used. The resin of olefin series has a low density and an acoustic impedance close to that of a living body, but has a high gas transparency (low gas barrier property). Gas, such as oxygen, passing from the side of the support layer oxidizes a Cr layer, which is for instance used as a adhesive layer for the Au film, and degrades the adhesive property.

The gas barrier layer 106 having a higher gas barrier property than the support layer 104 is a layer provided for preventing degradation of the adhesive property between the support layer 104 and the optical reflection layer 108 due to gas inflow. This gas barrier layer can suitably be made of material having a lower oxygen transparency than that of the resin of olefin series used as the support layer 104. An inorganic material can be selected as this material; SiO₂ (silicon oxide) and an SiN can be used. An SiO₂ film and an SiN film can be formed by sputtering. In this embodiment, the gas barrier layer 106 is disposed between the optical reflection layer 108 and the support layer 104 (i.e., the surface of the support layer 104 closer to the optical reflection layer 108). The gas barrier layer may be disposed between the support layer 300 and the acoustic impedance matching layer 303 (i.e., the surface of the support layer closer to the element), for instance, as illustrated in FIG. 3C. Instead, as illustrated in FIG. 4D, the gas barrier layers may be disposed between the optical reflection layer 402 and the support layer 400 and also between the support layer 400 and the acoustic impedance matching layer 404. The gas barrier layer 106 can suitably be thin in consideration of the acoustic impedance. More specifically, the gas barrier layer can suitably be 10 μm or less, and more suitably be 1 μm or less.

Examples of the present invention will hereinafter be described.

### EXAMPLE 1

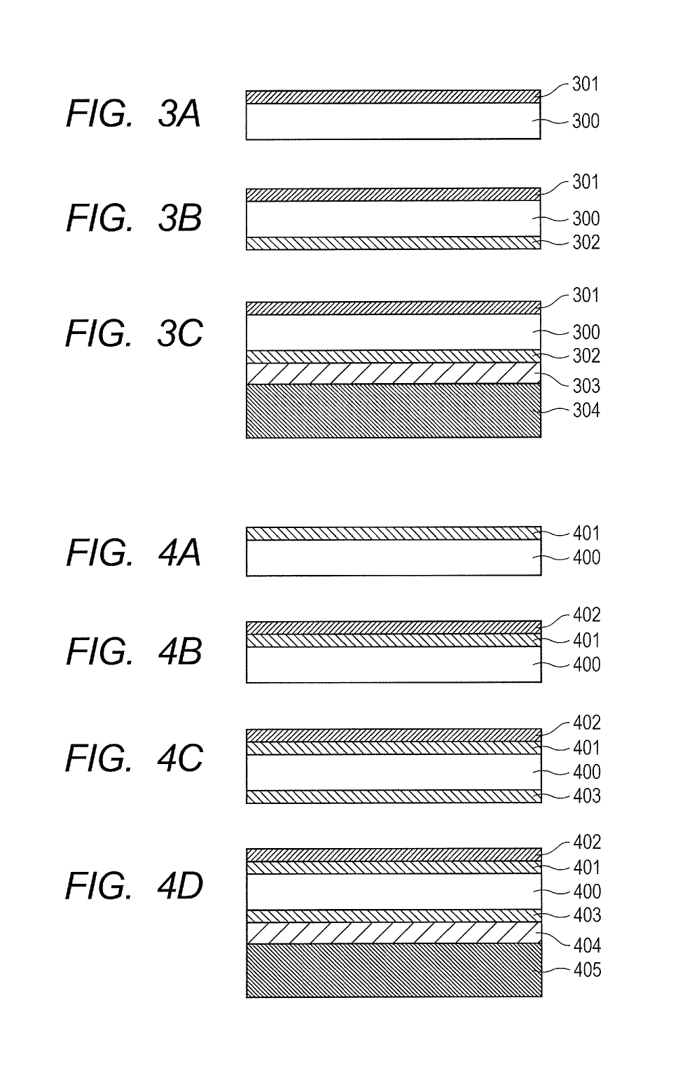

FIGS. 2A to 2C are sectional views illustrating a flow of steps of a method of manufacturing a photoacoustic probe of Example 1. As illustrated in FIG. 2A, 200 nm of SiO₂ is stacked by sputtering as a gas barrier layer 201 on a support substrate 200, which is to be a support layer as polymethylpentene resin having a thickness of 100 μm. Next, as illustrated in FIG. 2B, an optical reflection layer 202 is formed by sequentially stacking Cr (with a thickness of 10 nm), Au (with a thickness of 200 nm) on the gas barrier layer 201 using a sputtering method. Here, the Cr film is thus formed before the Au film is formed for improving the adhesive property to the gas barrier layer 201. After the gas barrier layer 201 and the optical reflection layer 202 are thus formed on the support layer 200, 40 μm of fluorosilicone resin (X-32-1619 made by Shin-Etsu Silicone) is applied by a printing method as an acoustic impedance matching layer 203 on the undersurface of the support layer 200. This layer is used as adhesive to cause the layers to adhere onto a CMUT substrate 204, as illustrated in FIG. 2C. Before the acoustic impedance matching layer 203 is applied on the support layer 200, an oxygen plasma process is applied to the application surface of the support layer 200 on which the acoustic impedance matching layer 203 is to be applied to improve the adhesive force between the support layer 200 and the acoustic impedance matching layer 203. The thus formed optical reflection layer of the photoacoustic probe favorably operates without causing film exfoliation.

### EXAMPLE 2

FIGS. 3A to 3C are diagrams illustrating another example of the present invention. First, as illustrated in FIG. 3A, an optical reflection layer 301 is formed by sequentially stacking Cr (with a thickness of 10 nm) and Au (with a thickness of 200 nm) using a sputtering method on a support layer 300 that is polymethylpentene resin having a thickness of 100 μm. Next, as illustrated in FIG. 3B, 200 nm of SiO₂ is stacked as a gas barrier layer 302 on the undersurface of the support layer 300 using a sputtering method. Thus, the optical reflection layer 301 is formed on the support layer 300, and the gas barrier layer 302 is formed on the undersurface of the support layer 300, and subsequently 40 μm of fluorosilicone resin (X-32-1619 made by Shin-Etsu Silicone) is applied as an acoustic impedance matching layer 303 on the gas barrier layer 302 using a printing method. This layer is used as adhesive to cause the layers to adhere onto a CMUT substrate 304 as illustrated in FIG. 3C. The thus formed optical reflection layer of the photoacoustic probe favorably operates without causing degradation, such as film exfoliation.

### EXAMPLE 3

FIGS. 4A to 4D are diagrams illustrating another example of the present invention. First, as illustrated in FIG. 4A, 200 nm of SiO₂ is stacked as a gas barrier layer 401 by sputtering on a support substrate 400, which is to be a support layer made of polymethylpentene resin having a thickness of 100 μm. SiN can be suitably used as the gas barrier layer. Next, as illustrated in FIG. 4B, an optical reflection layer 402 is formed by sequentially stacking Cr (with a thickness of 10 nm) and Au (with a thickness of 200 nm) using a sputtering method on the gas barrier layer 401. Here, before the Au film is formed, the Cr film is formed for improving adhesive property with the gas barrier layer 401. Thus, after the gas barrier layer 401 and the optical reflection layer 402 are formed on the support layer 400, 200 nm of SiO₂ is stacked as a gas barrier layer 403 using a sputtering method on the undersurface of the support layer 400 as illustrated in FIG. 4C. As described above, the gas barrier layer 401 and the optical reflection layer 402 are formed on the support layer 400. The gas barrier layer 403 is formed on the undersurface of the support layer 400. Subsequently, fluorosilicone resin (X-32-1619 made by Shin-Etsu Silicone) is applied into a thickness of 40 μm as an acoustic impedance matching layer 404 using a printing method on the gas barrier layer 403. This layer is used as adhesive to cause the layers to adhere onto a CMUT substrate 405 as illustrated in FIG. 4D. The thus formed optical reflection layer of the photoacoustic probe favorably operates without causing film exfoliation.

### EXAMPLE 4

The probe including the electromechanical transducer described in the embodiments and the examples is applicable to an object information acquisition apparatus using acoustic waves. Acoustic waves from an object are received by the electromechanical transducer. Through use of an output electric signal, object information in which an optical property value of the object, such as the optical absorption coefficient, is reflected can be acquired.

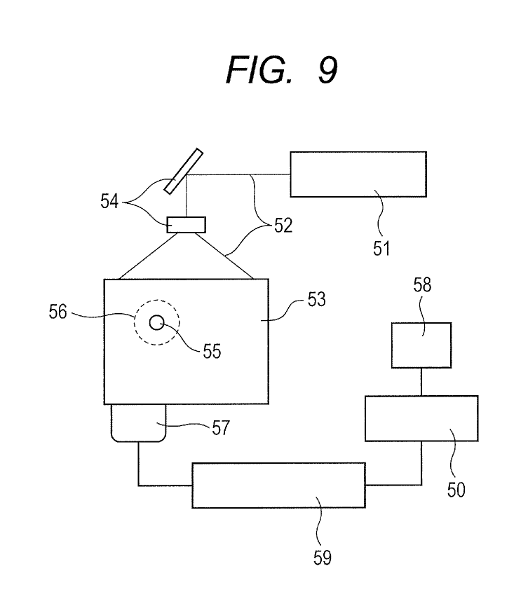

FIG. 9 illustrates an object information acquisition apparatus using photoacoustic effects according to this example. An object 53 is irradiated with pulsed light 52 emitted from a light source 51 via optical elements 54, such as a lens, a mirror and an optical fiber. A light absorber 55 in the object 53 absorbs the energy of the pulsed light and generates photoacoustic waves 56, which are acoustic waves. A probe 57 including a casing for accommodating an electromechanical transducer receives the photoacoustic waves 56, converts the waves into an electric signal and outputs the signal to a signal processor 59. The signal processor 59 performs a signal process, such as A/D conversion and amplification, on the input signal, and outputs the signal to a data processor 50. The data processor 50 acquires object information (object information in which an optical property value of the object, such as an optical absorption coefficient is reflected) as an image data, using the input signal. The display 58 displays an image based on the image data input from the data processor 50. The probe may be any of a type of being mechanically scanned and a type (hand-held type) of being moved by a user, such as any of a doctor and a technician, with respect to an object.

100: CMUT substrate, 102: acoustic impedance matching layer, 104: support layer, 106: gas barrier layer, 108: optical reflection layer, 110: optical reflection member

According to the present invention, on a support layer made of resin having a low acoustic impedance (e.g., resin of olefin series, such as methylpentene resin), a gas barrier layer that is made of SiO₂ and has a high gas barrier property is formed, and an optical reflection layer, such as a metal thin film, is formed thereon. Instead, a gas barrier layer having a high gas barrier property is formed between a support layer and an acoustic impedance matching layer. Accordingly, gas inflow into the adhesive interface due to a high gas transparency of resin used as the support layer can be suppressed. Thus, degradation of the adhesive property of the optical reflection layer due to variation of the state of the interface is suppressed, which can in turn suppress exfoliation of the optical reflection layer. Use of the support layer made of resin having a low acoustic impedance can realize the advantageous effects while suppressing reduction in sensitivity of the probe due to inconformity of the acoustic impedance.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-083417, filed on Mar. 31, 2012, and Japanese Patent Application No. 2013-040132, filed on Feb. 28, 2013, which are hereby incorporated by reference herein in its entirety.

What is claimed is:

1. A probe receiving an acoustic wave from an object, comprising:
an element having at least one cell in which a vibration film containing one electrode out of two electrodes that are provided so as to interpose a space therebetween is supported in a manner allowed to vibrate owing to the acoustic wave;
an optical reflection layer that is provided closer to the object than the element is;
a support layer that is provided closer to the element than the optical reflection layer is, and supports the optical reflection layer; and
a gas barrier layer that is provided on at least one of a surface of the support layer closer to the optical reflection layer and a surface of the support layer closer to the element and has a higher gas barrier property than the support layer.

2. The probe according to claim 1, further comprising an acoustic impedance matching layer between the support layer and the element.

3. The probe according to claim 1, wherein the gas barrier layer has a lower oxygen transmittance than the support layer.

4. The probe according to claim 1, wherein the gas barrier layer has a thickness equal to or less than 10 μm.

5. The probe according to claim 1, wherein an oxygen transmittance in a state where the gas barrier layer is formed on the support layer is equal to or less than 1×10⁻¹⁵ cm³·cm/(cm²·s·Pa).

6. The probe according to claim 1, wherein the gas barrier layer is one of an SiO₂ film and an SiN film.

7. The probe according to claim 1,
wherein the element receives an acoustic wave caused by irradiation with light on the object, and
the optical reflection layer has an optical reflectance of at least 80% in a wavelength region of the light.

8. The probe according to claim 1, wherein the optical reflection layer has a thickness equal to or less than 1/30 of a wavelength of the acoustic wave.

9. The probe according to claim 1, wherein the optical reflection layer is one of a metal film and a dielectric multilayer film, or has a layered structure of a metal film and a dielectric multilayer film.

10. The probe according to claim 1, wherein the support layer has an acoustic impedance between 1 and 5 MRayls, inclusive.

11. The probe according to claim 1, wherein the support layer has a higher Young's modulus than the acoustic impedance matching layer.

12. The probe according to claim 1, wherein the support layer has a Young's modulus between 100 MPa and 20 GPa, inclusive.

13. The probe according to claim 2, wherein the acoustic impedance matching layer has an acoustic impedance between 1 and 2 MRayls, inclusive.

14. The probe according to claim 2, wherein the acoustic impedance matching layer has a Young's modulus equal to or less than 50 MPa.

15. An object information acquisition apparatus, comprising: the probe according to claim 1; a light source; and a data processing device, wherein the probe receives an acoustic wave caused by irradiation on the object with light emitted from the light source and converts the wave into an electric signal, and
the data processing device acquires information on the object using the electric signal.
